SEQUENCE LISTING (12 sequences)

SEQ ID NO: 1 - protein, 118 aa, Artificial Sequence
  Description of Artificial Sequence Synthetic polypeptide
QVQLVQSGAEVKKPGSSVKVSCKASGYSFSDYYMHWVRQAPGQGLEWMGQINPTTGGASYNQKFKGKATITVDKSTSTAYMELSSLRSEDTAVYYCARYYYGRHFDVWGQGTTVTVSS

SEQ ID NO: 2 - protein, 10 aa, Artificial Sequence
  Description of Artificial Sequence Synthetic peptide
GYSFSDYYMH

SEQ ID NO: 3 - protein, 17 aa, Artificial Sequence
  Description of Artificial Sequence Synthetic peptide
QINPTTGGASYNQKFKG

SEQ ID NO: 4 - protein, 9 aa, Artificial Sequence
  Description of Artificial Sequence Synthetic peptide
YYYGRHFDV

SEQ ID NO: 5 - protein, 111 aa, Artificial Sequence
  Description of Artificial Sequence Synthetic polypeptide
DIVMTQSPDSLAVSLGERATINCKASQSVDYDGDSYMNWYQQKPGQPPKLLIYAASNLESGVPDRFSGSGSGTDFTLTISSLQAEDVAVYYCQQSNEDPYTFGQGTKLEIK

SEQ ID NO: 6 - protein, 15 aa, Artificial Sequence
  Description of Artificial Sequence Synthetic peptide
KASQSVDYDGDSYMN

SEQ ID NO: 7 - protein, 7 aa, Artificial Sequence
  Description of Artificial Sequence Synthetic peptide
AASNLES

SEQ ID NO: 8 - protein, 9 aa, Artificial Sequence
  Description of Artificial Sequence Synthetic peptide
QQSNEDPYT

SEQ ID NO: 9 - protein, 118 aa, Artificial Sequence
  Description of Artificial Sequence Synthetic polypeptide
QVQLQQSGPELVKPGASVKISCKASGYSFSDYYMHWVKQSPENSLEWIGQINPTTGGASYNQKFKGKATLTVDKSSSTAYMQLKSLTSEESAVYYCTRYYYGRHFDVWGQGTTVTVSS

SEQ ID NO: 10 - protein, 111 aa, Artificial Sequence
  Description of Artificial Sequence Synthetic polypeptide
DIVMTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIK

SEQ ID NO: 11 - protein, 450 aa, Artificial Sequence
  Description of Artificial Sequence Synthetic polypeptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 216 aa, Artificial Sequence
  Description of Artificial Sequence Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

(19) United States
(12) Patent Application Publication
(10) Pub. No.: US 2020/0317788 A1
(43) Pub. Date: Oct. 8, 2020

(54) SEMAPHORIN-4D ANTAGONISTS FOR USE IN CANCER THERAPY

(71) Applicant: Vaccinex, Inc., Rochester, NY (US)

(72) Inventors: Terrence Lee Fisher, Rochester, NY (US); Elizabeth Evans, Bloomfield, NY (US); Maurice Zauderer, Pittsford, NY (US)

(21) Appl. No.: 16/832,170

(22) Filed: Mar. 27, 2020

Related U.S. Application Data
(60) Provisional application No. 62/825,536, filed on Mar. 28, 2019.

(51) Int. Cl. C07K 16/28; G01N 33/49; A61P 35/00

(52) U.S. Cl. CPC C07K 16/2827; G01N 33/4915; A61K 45/06; C07K 2317/76; C07K 2317/565; A61P 35/00

(57) ABSTRACT

The disclosure relates to methods for treating cancer comprising determining certain patient biomarker levels prior to treatment.

### CROSS-REFERENCE TO RELATED APPLICATION

This is a non-provisional of pending U.S. provisional application Ser. No. 62/825,536, filed Mar. 28, 2019, the entirety of which application is incorporated by reference herein.

### SEQUENCE LISTING

The instant application contains a Sequence Listing which has been submitted electronically in ASCII format and is hereby incorporated by reference in its entirety. Said ASCII copy, created on Mar. 26, 2020, is named 8555_033_SL.txt and is 12,196 bytes in size.

Semaphorin 4D (SEMA4D), also known as CD100, is a transmembrane protein that belongs to the semaphorin gene family. SEMA4D is expressed on the cell surface as a homodimer, but upon cell activation SEMA4D can be released from the cell surface via proteolytic cleavage to generate sSEMA4D, a soluble form of the protein, which is also biologically active. See Suzuki et al., Nature Rev. Immunol. 3:159-167 (2003); Kikutani et al., Nature Immunol. 9:17-23 (2008).

SEMA4D is expressed at high levels in lymphoid organs, including the spleen, thymus, and lymph nodes, and in non-lymphoid organs, such as the brain, heart, and kidney. In lymphoid organs, SEMA4D is abundantly expressed on resting T cells but only weakly expressed on resting B cells and antigen-presenting cells (APCs), such as dendritic cells (DCs). Its expression, however, is upregulated in these cells following activation by various immunological stimuli. The release of soluble SEMA4D from immune cells is also increased by cell activation. SEMA4D has been implicated in the development of certain cancers (Ch'ng et al., Cancer 110:164-72 (2007); Campos et al., Oncology Letters, 5:1527-35 (2013); Kato et al., Cancer Sci. 102:2029-37 (2011)).

We have previously reported that the anti-SEMA4D antagonist monoclonal antibody VX15/2503 (pepinemab) is effective in treating a variety of cancers either alone (see, e.g., U.S. Pat. No. 9,605,055) or in combination with various other cancer immunotherapy treatments, including checkpoint blockades (see, e.g., U.S. Pat. No. 9,243,068). These results have been extended to the clinic, see e.g., Patnaik, A., et al. Clin. Cancer Res. 22:827-836 (2016). Moreover, we have shown that subjects with cancer tend to fare better when they have elevated levels of T cells, e.g., CD8+ T cells, B cells, or both T cells and B cells prior to treatment, relative to other cancer patients (see, e.g., U.S. Pat. No. 9,243,068).

Myeloid-derived suppressor cells (MDSC) are a heterogeneous group of myeloid origin cells with tumor promoting and/or immunosuppressive activities. See Lang, S., et al. Clin. Cancer Res. 24:4834-4844 (2018). Various populations of human MDSC are characterized by different surface markers. For example, circulating polymorphonuclear MDSC (PMN-MDSC) express CD15 and/or CD66b, lack the monocyte marker CD14, and are positive for CD33. Monocytic MDSC (M-MDSC) typically express higher levels of CD33 compared with PMN-MDSC, are CD14+, and can have low or even absent levels of HLA-DR. Id. MDSC can also be characterized by the absence of markers typical of other cell lineages, for example they can be characterized by the absence of the markers CD3, CD19, and/or CD56. See, e.g., Gabrilovich et al. Cancer Immunol Res. 5:3-8 (2017).

There remains a need in the art for additional methods of defining populations of cancer patients that are likely to benefit from treatment with pepinemab either alone or in combination with other immunotherapeutic agents.

### SUMMARY

The disclosure relates to methods for treating cancer and selecting subjects with cancer for treatment. The disclosure provides a method for treating and selecting subjects with cancer for treating, inhibiting, delaying, or reducing malignant cell growth in a subject by, administering to the subject an effective amount of a cancer immunotherapy regimen that includes administration of an isolated antibody or antigen-binding fragment thereof that specifically binds to semaphorin-4D (SEMA4D). The methods comprise determining the level of circulating MDSCs in the subject and selecting the subject for treatment if the level of MDSCs is below a predetermined threshold level. In certain aspects, the level of circulating MDSCs is determined by obtaining or having obtained a biological sample, such as a blood sample or a tumor biopsy from the subject and performing or having performed an assay, such as an immunophenotyping assay on the biological sample to determine the level of MDSCs in the biological sample. An effective amount of a cancer immunotherapy regimen comprising an isolated antibody or antigen-binding fragment thereof that specifically binds to semaphorin-4D (SEMA4D) is administered if the level of MDSCs is determined to be below a predetermined threshold level, thereby treating the subject. In certain aspects the anti-SEMA4D antibody or fragment thereof inhibits SEMA4D interaction with its receptor, e.g., Plexin-B1, Plexin-B2, CD72, or any combination thereof. In certain aspects administration of the antibody or fragment thereof inhibits SEMA4D-mediated signal transduction. In certain aspects the antibody or fragment thereof includes a variable heavy chain (VH) that includes VH CDRs 1-3 comprising SEQ ID NOS: 2, 3, and 4, respectively, and a variable light chain (VL) that includes VL CDRs 1-3 comprising SEQ ID NOS: 6, 7, and 8, respectively. In certain aspects the VH and VL include, respectively, the amino acid sequences SEQ ID NO: 1 and SEQ ID NO: 5, or SEQ ID NO: 9 and SEQ ID NO: 10.

In certain aspects the cancer immunotherapy regimen can further include an additional cancer immunotherapy agent, e.g., administration of an immune checkpoint blockade. The immune checkpoint blockade can include an antibody or antigen-binding fragment thereof that specifically binds to CTLA4, PD-1, PD-L1, LAG3, TIM3, B7-H3, or any combination thereof. In certain aspects the cancer immunotherapy regimen further includes administration of the anti-PD-L1 antibody avelumab.

In certain aspects the MDSCs are mononuclear MDSCs (M-MDSCs), e.g., MDSCs with a CD14⁺, HLA-DR^{-/low}, CD11b⁺, CD33⁺, Ln-phenotype, wherein Ln is a cocktail of markers that define non-MDSCs, e.g., the Ln markers can comprise one or more of CD3, CD19, or CD56. In certain aspects the predetermined threshold level of MDSCs comprises less than 50%, less than 40%, less than 30%, less than 20%, or less than 10% of the subject's total peripheral blood mononuclear cells prior to treatment.

In certain aspects the cancer can be a solid tumor, a hematological malignancy, any metastasis thereof, or any combination thereof. In certain aspects the cancer is non-small cell lung cancer.

### BRIEF DESCRIPTION OF THE DRAWINGS/FIGURES

FIG. 1A shows the level of CD14⁺, HLA-DR^{low}, CD11b⁺, CD33⁺, Ln⁻MDSC cells in subjects at the beginning of the study versus days on the study (for each subject, an average of a screening visit and baseline visit, expressed as a percentage of total peripheral blood lymphocytes), where the “Ln” phenotype excluded from the cell population includes CD3, CD19, and CD56.

FIG. 1B shows the level of CD8+ T cells in subjects at the beginning of the study versus days on the study (or each subject, an average of a screening visit and baseline visit, expressed cells per μl).

FIG. 1C compares the level of CD14⁺, HLA-DR^{low}, CD11b⁺, CD33⁺, Ln⁻MDSC cells in subjects at the beginning of the study versus the level of CD8+ T cells in subjects at the beginning of the study.

### DETAILED DESCRIPTION

### Definitions

It is to be noted that the term “a” or “an” entity refers to one or more of that entity; for example, “a binding molecule,” is understood to represent one or more binding molecules. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary of Biochemistry and Molecular Biology, 2d Edition, 2008, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. Accordingly, the terms defined immediately below are more fully defined by reference to the specification in its entirety.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, and derivatization by known protecting/blocking groups, proteolytic cleavage, or modification by non-naturally occurring amino acids. A polypeptide can be derived from a biological source or produced by recombinant technology but is not necessarily translated from a designated nucleic acid sequence. It can be generated in any manner, including by chemical synthesis.

Polypeptides can have a defined three-dimensional structure, although they do not necessarily have such structure. As used herein, the term glycoprotein refers to a protein coupled to at least one carbohydrate moiety that is attached to the protein via an oxygen-containing or a nitrogen-containing side chain of an amino acid, e.g., a serine or an asparagine.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are native or recombinant polypeptides which have been separated, fractionated, or partially or substantially purified by any suitable technique.

As used herein, the term “a non-naturally occurring polypeptide” or any grammatical variants thereof, is a conditional definition that explicitly excludes, but only excludes, those forms of the polypeptide that are, or might be, determined or interpreted by a judge or an administrative or judicial body, to be “naturally-occurring.”

Other polypeptides disclosed herein are fragments, derivatives, analogs, or variants of the foregoing polypeptides, and any combination thereof. The terms “fragment,” “variant,” “derivative” and “analog” as disclosed herein include any polypeptides which retain at least some of the properties of the corresponding native antibody or polypeptide, for example, specifically binding to an antigen. Fragments of polypeptides include, for example, proteolytic fragments, as well as deletion fragments, in addition to specific antibody fragments discussed elsewhere herein. Variants of, e.g., a polypeptide include fragments as described above, and also polypeptides with altered amino acid sequences due to amino acid substitutions, deletions, or insertions. Variant polypeptides can comprise conservative or non-conservative amino acid substitutions, deletions or additions. Derivatives are polypeptides that have been altered so as to exhibit additional features not found on the original polypeptide. Examples include fusion proteins.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the polypeptides and antibodies of the present disclosure do not abrogate the binding of the polypeptide or antibody containing the amino acid sequence, to the antigen to which the binding molecule binds. Methods of identifying nucleotide and amino acid conservative substitutions which do not eliminate antigen-binding are well-known in the art (see, e.g., Brummell et al., Biochem. 32: 1180-1 187 (1993); Kobayashi et al., Protein Eng. 12(10):879-884 (1999); and Burks et al., Proc. Natl. Acad. Sci. USA 94: 412-417 (1997)).

The term “polynucleotide” is intended to encompass a singular nucleic acid as well as plural nucleic acids and refers to an isolated nucleic acid molecule or construct, e.g., messenger RNA (mRNA), cDNA, or plasmid DNA (pDNA). A polynucleotide can comprise a conventional phosphodiester bond or a non-conventional bond (e.g., an amide bond, such as found in peptide nucleic acids (PNA)). The terms “nucleic acid” or “nucleic acid sequence” refer to any one or more nucleic acid segments, e.g., DNA or RNA fragments, present in a polynucleotide.

By an “isolated” nucleic acid or polynucleotide is intended any form of the nucleic acid or polynucleotide that is separated from its native environment. For example, gel-purified polynucleotide, or a recombinant polynucleotide encoding a polypeptide contained in a vector would be considered to be “isolated.” Also, a polynucleotide segment, e.g., a PCR product, which has been engineered to have restriction sites for cloning is considered to be “isolated.” Further examples of an isolated polynucleotide include recombinant polynucleotides maintained in heterologous host cells or purified (partially or substantially) polynucleotides in a non-native solution such as a buffer or saline. Isolated RNA molecules include in vivo or in vitro RNA transcripts of polynucleotides, where the transcript is not one that would be found in nature. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, the term “a non-naturally occurring polynucleotide” or any grammatical variants thereof, is a conditional definition that explicitly excludes, but only excludes, those forms of the nucleic acid or polynucleotide that are, or might be, determined or interpreted by a judge, or an administrative or judicial body, to be “naturally-occurring.”

As used herein, a “coding region” is a portion of nucleic acid which consists of codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region.

In certain embodiments, the polynucleotide or nucleic acid is DNA. In the case of DNA, a polynucleotide comprising a nucleic acid which encodes a polypeptide normally can include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA in predetermined cells. Other transcription control elements, besides a promoter, for example enhancers, operators, repressors, and transcription termination signals, can be operably associated with the polynucleotide to direct cell-specific transcription.

In other embodiments, a polynucleotide can be RNA, for example, in the form of messenger RNA (mRNA), transfer RNA, or ribosomal RNA.

As used herein, the term “binding molecule” refers in its broadest sense to a molecule that specifically binds to a receptor, e.g., an epitope or an antigenic determinant. As described further herein, a binding molecule can comprise one of more “antigen binding domains” described herein. A non-limiting example of a binding molecule is an antibody or fragment thereof that retains antigen-specific binding.

As used herein, the terms “binding domain” or “antigen-binding domain” refer to a region of a binding molecule that is necessary and sufficient to specifically bind to an epitope. For example, an “Fv,” e.g., a variable heavy chain and variable light chain of an antibody, either as two separate polypeptide subunits or as a single chain, is considered to be a “binding domain.” Other binding domains include, without limitation, the variable heavy chain (VHH) of an antibody derived from a camelid species, or six immunoglobulin complementarity determining regions (CDRs) expressed in a fibronectin scaffold.

An antibody (or an antigen-binding fragment, variant, or derivative thereof, or a multimeric fragment, variant, or derivative thereof, as disclosed herein) includes at least the variable domain of a heavy chain (for camelid species) or at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988). Unless otherwise stated, the term “antibody” encompasses anything ranging from a small antigen-binding fragment of an antibody to a full sized antibody, e.g., an IgG antibody that includes two complete heavy chains and two complete light chains.

As will be discussed in more detail below, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4 or α1-α2)). It is the nature of this chain that determines the “isotype” of the antibody as IgG, IgM, IgA IgG, or IgE, respectively. The immunoglobulin subclasses (subtypes) e.g., IgG₁, IgG₂, IgG₃, IgG₄, IgA₁, IgA₂, etc. are well characterized and are known to confer functional specialization. Modified versions of each of these immunoglobulins are readily discernible to the skilled artisan in view of the instant disclosure and, accordingly, are within the scope of this disclosure.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are expressed, e.g., by hybridomas, B cells or genetically engineered host cells. In the heavy chain, the amino acid sequences run from an N-terminus at the forked ends of the Y configuration to the C-terminus at the bottom of each chain. The basic structure of certain antibodies, e.g., IgG antibodies, includes two heavy chain subunits and two light chain subunits covalently connected via disulfide bonds to form a “Y” structure, also referred to herein as an “H2L2” structure.”

The term “epitope” includes any molecular determinant capable of specific binding to an antibody. In certain aspects, an epitope can include chemically active surface groupings of molecules such as amino acids, sugar side chains, phosphoryl, or sulfonyl, and, in certain aspects, can have a three-dimensional structural characteristics, and or specific charge characteristics. An epitope is a region of a target that is bound by an antibody.

The term “target” is used in the broadest sense to include substances that can be bound by a binding molecule. A target can be, e.g., a polypeptide, a nucleic acid, a carbohydrate, a lipid, or other molecule. Moreover, a “target” can, for example, be a cell, an organ, or an organism that comprises an epitope bound that can be bound by a binding molecule.

Both the light and heavy chains are divided into regions of structural and functional homology. The terms “constant” and “variable” are used functionally. In this regard, it will be appreciated that the variable domains of both the variable light (VL) and variable heavy (VH) chain portions determine antigen recognition and specificity. Conversely, the constant domains of the light chain (CL) and the heavy chain (e.g., CH1, CH2, CH3, or CH4 (where present)) confer biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 and CL domains actually comprise the carboxy-terminus of the heavy and light chain, respectively.

As indicated above, variable regions allow a binding molecule to selectively recognize and specifically bind epitopes on antigens. That is, the VL domain and VH domain, or subset of the complementarity determining regions (CDRs), of a binding molecule, e.g., an antibody, combine to form the antigen binding domain. More specifically, an antigen binding domain can be defined by three CDRs on each of the VH and VL chains.

The six “complementarity determining regions” or “CDRs” present in an antibody antigen-binding domain are short, non-contiguous sequences of amino acids that are specifically positioned to form the binding domain as the antibody assumes its three-dimensional configuration in an aqueous environment. The remainder of the amino acids in the binding domain, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The binding domain formed by the positioned CDRs defines a surface complementary to the epitope on the immunoreactive antigen. This complementary surface promotes the non-covalent binding of the antibody to its cognate epitope. The amino acids that make up the CDRs and the framework regions, respectively, can be readily identified for any given heavy or light chain variable region by one of ordinary skill in the art, since they have been defined in various different ways (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

The term “immunophenotyping assay” as used herein refers to a technique used to study the protein expressed by cells. This technique can be carried out on tissue section (fresh or fixed tissue), cell suspension, a blood sample, etc. A collection of immunophenotypic techniques and applications used in research and clinical settings is described in detail in Immunophenotyping: Methods and Protocols, McCoy, Jr., J. Philip (Ed.) (2019), incorporated herein by reference.

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. These particular regions have been described, for example, by Kabat et al., U.S. Dept. of Health and Human Services, “Sequences of Proteins of Immunological Interest” (1983) and by Chothia et al., J. Mol. Biol. 196:901-917 (1987), which are incorporated herein by reference. The Kabat and Chothia definitions include overlapping or subsets of amino acids when compared against each other. Nevertheless, application of either definition (or other definitions known to those of ordinary skill in the art) to refer to a CDR of an antibody or variant thereof is intended to be within the scope of the term as defined and used herein, unless otherwise indicated. The appropriate amino acids which encompass the CDRs as defined by each of the above cited references are set forth below in Table 1 as a comparison. The exact amino acid numbers which encompass a particular CDR will vary depending on the sequence and size of the CDR. Those skilled in the art can routinely determine which amino acids comprise a particular CDR given the variable region amino acid sequence of the antibody.

TABLE 1
CDR Definitions*

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

*Numbering of all CDR definitions in Table 1 is according to the numbering conventions set forth by Kabat et al. (see below).

Antibody variable domains can also be analyzed, e.g., using the IMGT information system (www://imgt.cines.fr/) (IMGT®/V-Quest) to identify variable region segments, including CDRs. (See, e.g., Brochet et al., Nucl. Acids Res., 36:W503-508, 2008).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless use of the Kabat numbering system is explicitly noted, however, consecutive numbering is used for all amino acid sequences in this disclosure.

Binding molecules, e.g., antibodies or antigen-binding fragments, variants, or derivatives thereof, or multimeric fragments, variants, or derivatives thereof include, but are not limited to, polyclonal, monoclonal, human, humanized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019.

By “specifically binds,” it is generally meant that a binding molecule, e.g., an antibody or fragment, variant, or derivative thereof binds to an epitope via its antigen binding domain, and that the binding entails some complementarity between the antigen binding domain and the epitope. According to this definition, a binding molecule is said to “specifically bind” to an epitope when it binds to that epitope, via its antigen binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

A binding molecule, e.g., an antibody or fragment, variant, or derivative thereof disclosed herein can be said to bind a target antigen with an off rate (k(off)) of less than or equal to 5×10⁻² sec⁻¹, 10⁻² sec⁻¹, 5×10⁻³ sec⁻¹, 10⁻³ sec⁻¹, 5×10⁻⁴ sec⁻¹, 10⁻⁴ sec⁻¹, 5×10⁻⁵ sec⁻¹, or 10⁻⁵ sec⁻¹, 5×10⁻⁶ sec⁻¹, 10⁻⁶ sec⁻¹, 5×10⁻⁷ sec⁻¹ or 10⁻⁷ sec⁻¹.

A binding molecule, e.g., an antibody or antigen-binding fragment, variant, or derivative disclosed herein can be said to bind a target antigen with an on rate (k(on)) of greater than or equal to 10³ M⁻¹ sec⁻¹, 5×10³ M⁻¹ sec⁻¹, 10⁴ M⁻¹ sec⁻¹, 5×10⁴ M⁻¹ sec⁻¹, 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

A binding molecule, e.g., an antibody or fragment, variant, or derivative thereof is said to competitively inhibit binding of a reference antibody or antigen binding fragment to a given epitope if it preferentially binds to that epitope to the extent that it blocks, to some degree, binding of the reference antibody or antigen binding fragment to the epitope. Competitive inhibition can be determined by any method known in the art, for example, competition ELISA assays. A binding molecule can be said to competitively inhibit binding of the reference antibody or antigen binding fragment to a given epitope by at least 90%, at least 80%, at least 70%, at least 60%, or at least 50%.

As used herein, the term “affinity” refers to a measure of the strength of the binding of an individual epitope with one or more binding domains, e.g., of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of binding domains and an antigen. See, e.g., Harlow at pages 29-34. Avidity is related to both the affinity of individual binding domains in the population with specific epitopes, and also the valencies of the immunoglobulins and the antigen. For example, the interaction between a bivalent monoclonal antibody and an antigen with a highly repeating epitope structure, such as a polymer, would be one of high avidity. An interaction between a between a bivalent monoclonal antibody with a receptor present at a high density on a cell surface would also be of high avidity.

Binding molecules, e.g., antibodies or antigen-binding fragments, variants or derivatives thereof as disclosed herein can also be described or specified in terms of their cross-reactivity. As used herein, the term “cross-reactivity” refers to the ability of a binding molecule, e.g., an antibody or fragment, variant, or derivative thereof, specific for one antigen, to react with a second antigen; a measure of relatedness between two different antigenic substances. Thus, a binding molecule is cross reactive if it binds to an epitope other than the one that induced its formation. The cross-reactive epitope generally contains many of the same complementary structural features as the inducing epitope, and in some cases, can actually fit better than the original.

A binding molecule, e.g., an antibody or fragment, variant, or derivative thereof can also be described or specified in terms of their binding affinity to an antigen. For example, a binding molecule can bind to an antigen with a dissociation constant or K_{D} no greater than 5×10⁻²M, 10⁻² M, 5×10⁻³M, 10⁻³M, 5×10⁻⁴M, 10⁻⁴M, 5×10⁻⁵M, 10⁻⁵M, 5×10⁻⁶M, 10⁻⁶M, 5×10⁻⁷M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³M, 10⁻¹³ M, 5×10⁻¹⁴ M, 10⁻¹⁴ M, 5×10⁻¹⁵ M, or 10⁻¹⁵ M.

As used herein the term “subunit” refers to a single polypeptide chain that combines with other identical or heterologous polypeptide chains to produce a binding molecule, e.g., an antibody or antigen-binding fragment thereof.

As used herein, the term “heavy chain subunit” includes amino acid sequences derived from an immunoglobulin heavy chain, a binding molecule, e.g., an antibody comprising a heavy chain subunit can include at least one of a VH domain, a CH1 domain, a hinge (e.g., upper, middle, and/or lower hinge region) domain, a CH2 domain, a CH3 domain, a CH4-tp domain, or a variant or fragment thereof.

As used herein, the term “light chain subunit” includes amino acid sequences derived from an immunoglobulin light chain. The light chain subunit includes at least a VL, and can further include a CL (e.g., Cκ or Cλ) domain.

Binding molecules, e.g., antibodies or antigen-binding fragments, variants, or derivatives thereof can be described or specified in terms of the epitope(s) or portion(s) of an antigen that they recognize or specifically bind. The portion of a target antigen that specifically interacts with the antigen binding domain of an antibody is an “epitope,” or an “antigenic determinant.” A target antigen can comprise a single epitope or at least two epitopes, and can include any number of epitopes, depending on the size, conformation, and type of antigen.

As used herein, the terms “cancer” and “cancerous” refer to or describe the physiological condition in mammals in which a population of cells are characterized by unregulated cell growth. Cancers can be categorized, e.g., as solid tumors or malignancies, or hematological cancers or malignancies. Both types can migrate to remote sites as metastases. A solid tumor can be categorized, e.g., as a sarcoma, a carcinoma, a melanoma, or a metastasis thereof.

The terms “proliferative disorder” and “proliferative disease” refer to disorders associated with abnormal cell proliferation such as cancer. “Tumor” and “neoplasm” as used herein refer to any mass of tissue that result from excessive cell growth or proliferation, either benign (noncancerous) or malignant (cancerous) including pre-cancerous lesions.

The terms “metastasis,” “metastases,” “metastatic,” and other grammatical equivalents as used herein refer to cancer cells which spread or transfer from the site of origin (e.g., a primary tumor) to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures. The terms also refer to the process of metastasis, which includes, but is not limited to detachment of cancer cells from a primary tumor, intravasation of the tumor cells to circulation, their survival and migration to a distant site, attachment and extravasation into a new site from the circulation, and microcolonization at the distant site, and tumor growth and development at the distant site.

Examples of such solid tumors can include, e.g., squamous cell carcinoma, adenocarcinoma, basal cell carcinoma, renal cell carcinoma, ductal carcinoma of the breast, soft tissue sarcoma, osteosarcoma, melanoma, small-cell lung cancer, non-small cell lung cancer (NSCLC), adenocarcinoma of the lung, cancer of the peritoneum, hepatocellular carcinoma, gastrointestinal cancer, gastric cancer, pancreatic cancer, neuroendocrine cancer, glioblastoma, cervical cancer, ovarian cancer, liver cancer, bladder cancer, brain cancer, hepatoma, breast cancer, colon cancer, colorectal cancer, endometrial or uterine carcinoma, esophageal cancer, salivary gland carcinoma, kidney cancer, prostate cancer, vulval cancer, thyroid cancer, head and neck cancer, any metastases thereof, or any combination thereof.

Examples of hematologic cancers or malignancies include without limitation leukemia, lymphoma, myeloma, acute myeloid leukemia, chronic myeloid leukemia, acute lymphocytic leukemia, chronic lymphocytic leukemia, hairy cell leukemia, Hodgkin lymphoma, non-Hodgkin lymphoma, multiple myeloma, any metastases thereof, or any combination thereof.

In certain embodiments, cancers that are amenable to treatment via the methods provided herein include, but are not limited to sarcomas, breast carcinomas, ovarian cancer, cervical cancer, head and neck cancer, NSCLC, esophageal cancer, gastric cancer, kidney cancer, liver cancer, bladder cancer, colorectal cancer, and pancreatic cancer.

The term “immune modulating agent” refers to the active agents of immunotherapy. Immune modulating agents include a diverse array of recombinant, synthetic and natural, preparation. Examples of immune modulating agents include, but are not limited to, interleukins such as IL-2, IL-7, IL-12; cytokines such as granulocyte colony-stimulating factor (G-CSF), interferons; various chemokines such as CXCL13, CCL26, CXCL7; antagonists of immune checkpoint blockades such as anti-CTLA-4, anti-PD-1 or anti-PD-L1 (ligand of PD-1), anti-LAG3, anti-B7-H3, synthetic cytosine phosphate-guanosine (CpG) oligodeoxynucleotides, glucans; and modulators of regulatory T cells (Tregs) such as cyclophosphamide.

The term “therapeutically effective amount” refers to an amount of an antibody, polypeptide, polynucleotide, small organic molecule, or other drug effective to “treat” or in some instances, “prevent” a disease or disorder in a subject, e.g., a human. In the case of cancer, the therapeutically effective amount of the drug can reduce the number of cancer cells; retard or stop cancer cell division, reduce or retard an increase in tumor size; inhibit, e.g., suppress, retard, prevent, stop, delay, or reverse cancer cell infiltration into peripheral organs including, for example, the spread of cancer into soft tissue and bone; inhibit, e.g., suppress, retard, prevent, shrink, stop, delay, or reverse tumor metastasis; inhibit, e.g., suppress, retard, prevent, stop, delay, or reverse tumor growth; relieve to some extent one or more of the symptoms associated with the cancer, reduce morbidity and mortality; improve quality of life; or a combination of such effects. To the extent the drug prevents growth and/or kills existing cancer cells, it can be referred to as cytostatic and/or cytotoxic.

Terms such as “treating” or “treatment” or “to treat” or “alleviating” or “to alleviate” refer to both 1) therapeutic measures that cure, slow down, lessen symptoms of, reverse, and/or halt progression of a diagnosed pathologic condition or disorder and 2) prophylactic or preventative measures that prevent and/or slow the development of a targeted pathologic condition or disorder. Thus, those in need of treatment include those already with the disorder; those prone to have the disorder; and those in whom the disorder is to be prevented. A subject is successfully “treated” according to the methods of the present disclosure if the patient shows one or more of the following: a reduction in the number of or complete absence of cancer cells; a reduction in the tumor size; or retardation or reversal of tumor growth, inhibition, e.g., suppression, prevention, retardation, shrinkage, delay, or reversal of metastases, e.g., of cancer cell infiltration into peripheral organs including, for example, the spread of cancer into soft tissue and bone; inhibition of, e.g., suppression of, retardation of, prevention of, shrinkage of, reversal of, delay of, or an absence of tumor metastases; inhibition of, e.g., suppression of, retardation of, prevention of, shrinkage of, reversal of, delay of, or an absence of tumor growth; relief of one or more symptoms associated with the specific cancer; reduced morbidity and mortality; improvement in quality of life; or some combination of effects. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, swine, cows, bears, and so on.

As used herein, phrases such as “a subject that would benefit from therapy” and “an animal in need of treatment” includes subjects, such as mammalian subjects, that would benefit from administration of a binding molecule such as an antibody, comprising one or more antigen binding domains. Such binding molecules, e.g., antibodies, can be used, e.g., for a diagnostic procedures and/or for treatment or prevention of a disease.

### Target Polypeptide Description—SEMA4D

As used herein, the terms “semaphorin-4D”, “SEMA4D”, and “SEMA4D polypeptide” are used interchangeably, as are “SEMA4D” and “Sema4D.” In certain embodiments, SEMA4D is membrane bound. In other embodiments, SEMA4D is soluble, e.g., sSEMA4D. In other embodiments, SEMA4D can include a full-sized SEMA4D or a fragment thereof, or a SEMA4D variant polypeptide, wherein the fragment of SEMA4D or SEMA4D variant polypeptide retains some or all functional properties of the full-sized SEMA4D.

The full-sized human SEMA4D protein is a homodimeric transmembrane protein consisting of two polypeptide chains of 150 kDa. SEMA4D belongs to the semaphorin family of cell surface receptors and is also referred to as CD100. Both human and mouse SEMA4D/Sema4D are proteolytically cleaved from their transmembrane form to generate 120-kDa soluble forms, giving rise to two Sema4D isoforms (Kumanogoh et al., J. Cell Science 116(7):3464 (2003)). Semaphorins consist of soluble and membrane-bound proteins that were originally defined as axonal-guidance factors which play an important role in establishing precise connections between neurons and their appropriate target.

SEMA4D is known to have at least three functional receptors, Plexin-B1, Plexin-B2 and CD72. Plexin-B1, is expressed in non-lymphoid tissues and has been shown to be a high affinity (1 nM) receptor for SEMA4D (Tamagnone et al., Cell 99:71-80 (1999)). Plexin-B2 has an intermediate affinity for SEMA4D and a recent report indicates that PLXNB2 is expressed on keratinocytes and activates SEMA4D-positive γδ T cells to contribute to epithelial repair (Witherden et al., Immunity I(2):314-25 (2012)). In lymphoid tissues, CD72 is utilized as a low affinity (300 nM) SEMA4D receptor (Kumanogoh et al., Immunity 13:621-631 (2000)).

SEMA4D is expressed at high levels in lymphoid organs, including the spleen, thymus, and lymph nodes, and in non-lymphoid organs, such as the brain, heart, and kidney. In lymphoid organs, SEMA4D is abundantly expressed on resting T cells but only weakly expressed on resting B cells and antigen-presenting cells (APCs), such as dendritic cells (DCs). Cellular activation increases the surface expression of SEMA4D as well as the generation of soluble SEMA4D (sSEMA4D).

### Anti-SEMA4D Antibodies

Antibodies that bind SEMA4D have been described in the art. See, for example, U.S. Pat. Nos. 7,919,594, 8,496,938, 8,816,058, 9,605,055, 9,676,840, 9,243,068, and 9,828,435, International Patent Application WO 93/14125, and Herold et al., Int. Immunol. 7(1): 1-8 (1995), each of which is herein incorporated in its entirety by reference.

The disclosure generally relates to a method of treating and selecting subjects with cancer for treatment to inhibit, delay, or reduce tumor growth or metastases in the subject, e.g., a human cancer patient, comprising determining the level of circulating MDSCs in the subject and selecting the subject for treatment if the level of MDSCs is below a predetermined threshold level. The level of circulating MDSCs may be determined by obtaining or having obtained a biological sample from the subject, such as a blood sample or a tumor biopsy and performing or having performed an assay such as an immunophenotyping assay on the biological sample to determine the level of MDSCs in the biological sample. An effective amount of a cancer immunotherapy regimen comprising an isolated antibody or antigen-binding fragment, variant or derivative thereof that specifically binds to semaphorin-4D (SEMA4D) is administered to the subject if the level of MDSCs is determined to be below a predetermined threshold level, thereby treating the subject. In certain embodiments, the antibody blocks the interaction of SEMA4D with one or more of its receptors, e.g., Plexin-B1 and/or Plexin-B2. In certain embodiments the cancer cells express Plexin-B1 and/or Plexin-B2. Anti-SEMA4D antibodies having these properties can be used in the methods provided herein. Antibodies that can be used include, but are not limited to MAbs VX15/2503, 67, 76, 2282 and antigen-binding fragments, variants, or derivatives thereof which are fully described, e.g., in U.S. Pat. No. 8,496,938. Additional antibodies which can be used in the methods provided herein include the BD16 antibody described in US 2006/0233793 A1 as well as antigen-binding fragments, variants, or derivatives thereof, or any of MAb 301, MAb 1893, MAb 657, MAb 1807, MAb 1656, MAb 1808, Mab 59, MAb 2191, MAb 2274, MAb 2275, MAb 2276, MAb 2277, MAb 2278, MAb 2279, MAb 2280, MAb 2281, MAb 2282, MAb 2283, MAb 2284, and MAb 2285, as well as any fragments, variants or derivatives thereof as described in U.S. Pat. No. 7,919,594. In certain embodiments an anti-SEMA4D antibody for use in the methods provided herein binds human, murine, or both human and murine SEMA4D. Also useful are antibodies which bind to the same epitope as any of the aforementioned antibodies and/or antibodies which competitively inhibit binding or activity of any of the aforementioned antibodies.

In certain aspects the anti-SEMA4D antibody or antigen-binding fragment thereof comprises the six CDRs of murine antibody MAb 67 and the humanized antibody VX15/2503, which, as a human IgG4 antibody is referred to in the art as pepinemab. The variable heavy chain (VH) of these antibodies comprises VH CDRs 1-3 comprising SEQ ID NOS: 2, 3, and 4, respectively, and the variable light chain (VL) comprises VL CDRs 1-3 comprising SEQ ID NOS: 6, 7, and 8, respectively. In certain aspects, the antibody comprises humanized VH and VL regions comprising the amino acid sequences SEQ ID NO: 1 and SEQ ID NO: 5, respectively. In certain aspects the antibody comprises murine VH and VL regions comprising the amino acid sequences SEQ ID NO: 9 and SEQ ID NO: 10, respectively.

Treatment Methods Using Therapeutic Anti-SEMA4D Antibodies as a Single Agent or in Combination with at Least One Immune Modulating Therapy

Methods of the disclosure are directed to the use of SEMA4D antagonists, e.g., anti-SEMA4D antibodies or antigen-binding fragments, variants, and derivatives thereof, either as single agents or in combination with at least one other immune modulating therapy, to inhibit, delay, or reduce tumor growth or metastases in a subject in need of such inhibition, delay, or reduction, e.g., a cancer patient. In certain aspects provided herein, subjects to be treated include those who have reduced levels of MDSCs prior to treatment, e.g., MDSC levels below a certain threshold level, e.g., in peripheral blood or in the tumor microenvironment.

In one aspect, the disclosure provides a method for selecting a subject with cancer for treating, inhibiting, delaying, or reducing malignant cell growth in the subject, comprising: determining the subject's level of circulating myeloid-derived suppressor cells (MDSCs) and administering to the subject an effective amount of a cancer immunotherapy regimen comprising a SEMA4D antagonist, e.g., an isolated antibody or antigen-binding fragment thereof that specifically binds to SEMA4D, if the level of MDSCs is below a predetermined threshold level, thereby treating the subject.

MDSCs can be measured by any known method, and the levels can be expressed as absolute numbers of cells, e.g., in peripheral blood or in the tumor microenvironment, or as a percentage of peripheral blood cells, or as a percentage of a sub-population of peripheral blood cells. Cells are typically measured by flow cytometry as described elsewhere herein. By “a predetermined threshold level” is meant that the level of MDSC cells measured in the subject are below a defined level, e.g., below the average level seen in comparable cancer patients or equivalent to or below the level typically measured in normal healthy donors. In certain aspects the “predetermined threshold level” can be a specific absolute number of MDSCs in, e.g., the peripheral blood or tumor microenvironment, or a percentage of a population of cells, e.g., the percentage of MDSCs in total peripheral blood mononuclear cells. In certain aspects the predetermined threshold level of MDSCs comprises less than 50%, less than 40%, less than 30%, less than 20%, or less than 10% of the subject's total peripheral blood mononuclear cells prior to treatment.

In certain aspects the MDSCs are mononuclear MDSCs (M-MDSCs). In certain aspects the M-MDSCs comprise a phenotype of cell surface markers. For example, certain populations of M-MDSCs express CD14, CD11b, and CD33, but express no or only low levels of the HLA-DR marker. Cells expressing certain cell surface markers, e.g., CD3, CD19, and CD56, can be excluded from the MDSC population. In certain aspects the M-MDSCs comprise a CD14⁺, HLA-DR^{-/low}, CD11b⁺, CD33⁺, Ln⁻ phenotype, wherein Ln is a cocktail of markers that define non-MDSCs. A typical cocktail includes, but is not limited to, any combination of CD3, CD19, and/or CD56. In certain aspects the M-MDSCs express CD14 and high levels of HLA-DR, but do not express CD16 (see Krieg et al., Nature Med. 24:144-154 (2018)). In certain aspects the MDSCs are polymorphonuclear MDSCs (PMN-MDSCs) expressing CD15 CD66b, and/or CD33, but not expressing CD14. Other MDSC phenotypes will be readily apparent to those of ordinary skill in the art.

In certain aspects the anti-SEMA4D antibody or fragment thereof administered as part of the cancer immunotherapy regimen inhibits SEMA4D interaction with its receptor, e.g., Plexin-B1, Plexin-B2, CD72, or any combination thereof. In certain aspects the anti-SEMA4D antibody or fragment thereof administered as part of the cancer immunotherapy regimen inhibits SEMA4D-mediated signal transduction. Suitable anti-SEMA4D antibodies are disclosed elsewhere herein and include, but are not limited to, pepinimab.

In certain aspects the cancer immunotherapy regimen is a combination treatment, and further includes administration of an additional cancer immunotherapy agent which can be, e.g., at least one immune modulatory agent. Suitable immunotherapy and immunomodulatory agents are described elsewhere herein. In certain aspects the additional cancer immunotherapy agent is an immune checkpoint blockade, e.g., an antibody or antigen-binding fragment thereof that specifically binds to CTLA4, PD-1, PD-L1, LAG3, TIM3, B7-H3, or any combination thereof. In certain aspects the checkpoint blockade antibody is the anti-PD-L1 antibody avelumab.

The provided method can be used to select and treat subjects with any cancer, e.g., a solid tumor, a hematological malignancy, any metastasis thereof, or any combination thereof. In certain aspect the solid tumor is a sarcoma, a carcinoma, a melanoma, any metastases thereof, or any combination thereof. In certain aspects the solid tumor can be squamous cell carcinoma, adenocarcinoma, basal cell carcinoma, renal cell carcinoma, ductal carcinoma of the breast, soft tissue sarcoma, osteosarcoma, melanoma, small-cell lung cancer, non-small cell lung cancer, adenocarcinoma of the lung, cancer of the peritoneum, hepatocellular carcinoma, gastrointestinal cancer, gastric cancer, pancreatic cancer, neuroendocrine cancer, glioblastoma, cervical cancer, ovarian cancer, liver cancer, bladder cancer, brain cancer, hepatoma, breast cancer, colon cancer, colorectal cancer, endometrial or uterine carcinoma, esophageal cancer, salivary gland carcinoma, kidney cancer, liver cancer, prostate cancer, vulval cancer, thyroid cancer, head and neck cancer, any metastases thereof, or any combination thereof. In certain aspects the cancer is non-small cell lung cancer. In certain aspects the hematologic malignancy is leukemia, lymphoma, myeloma, acute myeloid leukemia, chronic myeloid leukemia, acute lymphocytic leukemia, chronic lymphocytic leukemia, hairy cell leukemia, Hodgkin lymphoma, non-Hodgkin lymphoma, multiple myeloma, any metastases thereof, or any combination thereof.

The method provided by this disclosure can further include administration of an additional cancer therapy, including, but not limited to surgery, chemotherapy, radiation therapy, administration of a cancer vaccine, administration of an immunostimulatory agent, adoptive T cell therapy, administration of a regulatory T cell (Treg) modulator, or any combination thereof.

In certain aspects the cancer cells, or cells in the vicinity of the cancer cells, express a SEMA4D receptor, in certain embodiments the receptor is Plexin-B1. Though the following discussion refers to administration of an anti-SEMA4D antibody, the methods described herein are equally applicable to any SEMA4D antagonist, i.e., an agent that inhibits the interaction of SEMA4D with one of its receptors, including, e.g., antigen-binding fragments, variants, and derivatives of anti-SEMA4D antibodies that retain the desired properties of the antibodies of the disclosure, e.g., capable of specifically binding SEMA4D, e.g., human, mouse, or human and mouse SEMA4D, having SEMA4D neutralizing/antagonist activity, and/or blocking the interaction of SEMA4D with any one or more of its receptors. The methods described herein are also applicable to other biologic products or small molecule drugs that retain the desired properties of a SEMA4D antagonist, e.g., capable of specifically binding SEMA4D, e.g., human, mouse, or human and mouse SEMA4D, having SEMA4D neutralizing/antagonist activity, and/or blocking the interaction of SEMA4D with its receptors.

In one embodiment, a SEMA4D antagonist, e.g., an anti-SEMA4D antibody or fragment, variant, or derivative thereof can be used as a single agent to inhibit, delay, or reduce tumor growth in a subject in need of such inhibition, delay, or reduction, e.g., a cancer patient, where in certain aspects, the subject is identified as a subject having MDSCs below a certain threshold level prior to treatment. In other aspects, a SEMA4D antagonist, e.g., an anti-SEMA4D antibody or fragment, variant, or derivative thereof can be administered in combination with other cancer therapies, including cancer immunotherapies such as, but not limited to cancer vaccines, immunostimulatory agents, adoptive T cell or antibody therapy, and immune checkpoint inhibitors.

Cancer Vaccines. Cancer vaccines activate the body's immune system and natural resistance to an abnormal cell, such as cancer, resulting in eradication or control of the disease. Cancer vaccines generally consist of a tumor antigen in an immunogenic formulation that activates tumor antigen-specific helper cells and/or CTLs and B cells. Vaccines can be in a variety of formulations, including, but not limited to, dendritic cells, especially autologous dendritic cells pulsed with tumor cells or tumor antigens, heterologous tumor cells transfected with an immune stimulating agent such as GM-CSF, recombinant virus, or proteins or peptides that are usually administered together with a potent immune adjuvant such as CpG.

Immunostimulatory Agents.

Immunostimulatory agents act to enhance or increase the immune response to tumors, which is suppressed in many cancer patients through various mechanisms. Immune modulating therapies can target lymphocytes, macrophages, dendritic cells, natural killer cells (NK Cell), or subsets of these cells such as cytotoxic T lymphocytes (CTL) or Natural Killer T (NKT) cells. Because of interacting immune cascades, an effect on one set of immune cells will often be amplified by spreading to other cells, e.g. enhanced antigen presenting cell activity promotes response of T and B lymphocytes. Examples of immunostimulatory agents include, but are not limited to, HER2, cytokines such as G-CSF, GM-CSF and IL-2, cell membrane fractions from bacteria, glycolipids that associate with CD1 d to activate Natural Killer T (NKT) cells, CpG oligonucleotides.

Macrophages, myelophagocytic cells of the immune system, are a fundamental part of the innate defense mechanisms, which can promote specific immunity by inducing T cell recruitment and activation. Despite this, their presence within the tumor microenvironment has been associated with enhanced tumor progression and shown to promote cancer cell growth and spread, angiogenesis and immunosuppression. Key players in the setting of their phenotype are the microenvironmental signals to which macrophages are exposed, which selectively tune their functions within a functional spectrum encompassing the M1 (tumor inhibiting macrophage) and M2 (tumor promoting macrophage) extremes. Sica et al., Seminars in Cancer Biol. 18:349-355 (2008). Increased macrophage numbers during cancer generally correlates with poor prognosis (Qualls and Murray, Curr. Topics in Develop. Biol. 94:309-328 (2011)). Of the multiple unique stromal cell types common to solid tumors, tumor-associated macrophages (TAMs) are significant for fostering tumor progression. Targeting molecular pathways regulating TAM polarization holds great promise for anticancer therapy. Ruffell et al., Trends in Immunol. 33:119-126 (2012).

Adoptive Cell Transfer.

Adoptive cell transfer can employ T cell-based cytotoxic responses to attack cancer cells. Autologous T cells that have a natural or genetically engineered reactivity to a patient's cancer are generated and expanded in vitro and then transferred back into the cancer patient. One study demonstrated that adoptive transfer of in vitro expanded autologous tumor-infiltrating lymphocytes was an effective treatment for patients with metastatic melanoma. (Rosenberg S A, Restifo N P, Yang J C, Morgan R A, Dudley M E (April 2008). Nat. Rev. Cancer 8 (4): 299-308). This can be achieved by taking T cells that are found within resected patient tumor. These T cells are referred to as tumor-infiltrating lymphocytes (TIL) and are presumed to have trafficked to the tumor because of their specificity for tumor antigens. Such T cells can be induced to multiply in vitro using high concentrations of IL-2, anti-CD3 and allo-reactive feeder cells. These T cells are then transferred back into the patient along with exogenous administration of IL-2 to further boost their anti-cancer activity. In other studies, autologous T cells have been transduced with a chimeric antigen receptor (“CAR-T cells”) that renders them reactive to a targeted tumor antigen (see, e.g., Liddy et al., Nature Med. 18:980-7, (2012); Grupp et al., New England J. Med. 368:1509-18, (2013); Petitt, et al., Mol Ther. 26:342-353 (2018)).

Other adoptive cell transfer therapies employ autologous dendritic cells exposed to natural or modified tumor antigens ex vivo that are re-infused into the patient. Provenge is such an FDA approved therapy in which autologous cells are incubated with a fusion protein of prostatic acid phosphatase and GM-CSF to treat patients with prostate tumors. GM-CSF is thought to promote the differentiation and activity of antigen presenting dendritic cells (Small et al., J. Clin. Oncol. 18: 3894-903(2000); U.S. Pat. No. 7,414,108)).

Immune Checkpoint Inhibitors.

Immune checkpoint inhibitor therapies enhance T-cell immunity by removing a negative feedback control that limits ongoing immune responses. These types of therapies target inhibitory pathways in the immune system that are crucial for modulating the duration and amplitude of physiological immune responses in peripheral tissues (anti-CTLA4) or in tumor tissue expressing PD-L1 (anti-PD-1 or anti-PD-L1) in order to minimize collateral tissue damage. Tumors can evolve to exploit certain immune-checkpoint pathways as a major mechanism of immune resistance against T cells that are specific for tumor antigens. Since many immune checkpoints are initiated by ligand-receptor interactions, these checkpoints can be blocked by antibodies to either receptor or ligand or can be modulated by soluble recombinant forms of the ligands or receptors. Neutralization of immune checkpoints allows tumor-specific T cells to continue to function in the otherwise immunosuppressive tumor microenvironment. Examples of immune checkpoint blockade therapies are those which target Cytotoxic T-lymphocyte-associated antigen 4 (CTLA-4), PD-1, its ligand PD-L1, LAG3 and B7-H3.

Cyclophosphamide.

Cyclophosphamide, a commonly used chemotherapeutic agent, can enhance immune responses. Cyclophosphamide differentially suppresses the function of regulatory T cells (Tregs) relative to effector T cells. Tregs are important in regulating anticancer immune responses. Tumor-infiltrating Tregs have previously been associated with poor prognosis. While agents that target Tregs specifically are currently unavailable, cyclophosphamide has emerged as a clinically feasible agent that can preferentially suppress Tregs relative to other T cells and, therefore, allows more effective induction of antitumor immune responses.

Other Immune-Modulating Therapies.

In another embodiment, therapy with a SEMA4D antagonist, e.g., a SEMA4D antibody or antigen binding fragment, variant, or derivative thereof, can be combined with either low dose chemotherapy or radiation therapy. Although standard chemotherapy is often immunosuppressive, low doses of chemotherapeutic agents such as cyclophosphamide, doxorubicin, and paclitaxel have been shown to enhance responses to vaccine therapy for cancer (Machiels et al., Cancer Res. 61:3689-3697 (2001)). In some cases, chemotherapy can differentially inactivate T regulatory cells (Treg) and myeloid derived suppressor cells (MDSC) that negatively regulate immune responses in the tumor environment. Radiation therapy has been generally employed to exploit the direct tumorcidal effect of ionizing radiation. Indeed, high dose radiation can, like chemotherapy, be immunosuppressive. Numerous observations, however, suggest that under appropriate conditions of dose fractionation and sequencing, radiation therapy can enhance tumor-specific immune responses and the effects of immune modulating agents. One of several mechanisms that contribute to this effect is cross-presentation by dendritic cells and other antigen presenting cells of tumor antigens released by radiation-induced tumor-cell death (Higgins et al., Cancer Biol. Ther. 8:1440-1449 (2009)). In effect, radiation therapy can induce in situ vaccination against a tumor (Ma et al., Seminar Immunol. 22:113-124 (2010)) and this could be amplified by combination with therapy with a SEMA4D antagonist, e.g., a SEMA4D antibody or antigen binding fragment, variant, or derivative thereof.

In one embodiment, the immune modulating therapy can be an immune modulating agent, including, but not limited to, interleukins such as IL-2, IL-7, IL-12; cytokines such as granulocyte-macrophage colony-stimulating factor (GM-CSF), interferons; various chemokines such as CXCL13, CCL26, CXCL7; antagonists of immune checkpoint blockades such as anti-CTLA-4, anti-PD-1, anti-PD-L1, anti-LAG3 and anti-B7-H3; synthetic cytosine phosphate-guanosine (CpG), oligodeoxynucleotides, glucans, modulators of regulatory T cells (Tregs) such as cyclophosphamide, or other immune modulating agents. In one embodiment, the immune modulating agent is an agonist antibody to 4-1BB (CD137). As recently reported, such agonist antibody to 4-1BB can give rise to a novel class of KLRG1+ T cells that are highly cytotoxic for tumors (Curran et al., J. Exp. Med. 210:743-755 (2013)). In all cases, the additional immune modulating therapy is administered prior to, during, or subsequent to the SEMA4D antagonist, e.g., the anti-SEMA4D antibody or antigen binding fragment, variant, or derivative thereof, therapy. Where the combined therapies comprise administration of an anti-SEMA4D binding molecule, e.g., an antibody or antigen binding fragment, variant, or derivative thereof, in combination with administration of another immune modulating agent, the methods of the disclosure encompass co-administration, using separate formulations or a single pharmaceutical formulation, with simultaneous or consecutive administration in either order.

In one embodiment, the immune modulating therapy can be a cancer therapy agent, including, but not limited to, surgery or surgical procedures (e.g. splenectomy, hepatectomy, lymphadenectomy, leukophoresis, bone marrow transplantation, and the like); radiation therapy; chemotherapy, optionally in combination with autologous bone marrow transplant, or other cancer therapy; where the additional cancer therapy is administered prior to, during, or subsequent to the SEMA4D antagonist, e.g., the anti-SEMA4D antibody or antigen binding fragment, variant, or derivative thereof, therapy. Where the combined therapies comprise administration of an anti-SEMA4D antibody or antigen binding fragment, variant, or derivative thereof, in combination with administration of another therapeutic agent, the methods of the disclosure encompass co-administration, using separate formulations or a single pharmaceutical formulation, with simultaneous or consecutive administration in either order.

In another embodiment, the disclosure is directed to the use of a SEMA4D antagonist, e.g., an anti-SEMA4D antibody or antigen-binding fragment, variant, or derivative thereof either as single agents or in combination with at least one other immune modulating therapy, to treat cancer patients with reduced levels of MDSCs in circulation prior to treatment, e.g., below a predetermined threshold level, when compared to other patients with solid tumors, such as those found in the brain, lung, ovary, breast, colon and other tissues, or other patients with hematological cancers. As used herein, the term “reduced” refers to cancer patients that have less than 90%, less than 80%, less than 70%, less than 60%, less than 50%, less than 40%, less than 30%, or less that 20% of the mean number of MDSCs in circulation than other cancer patients. The number of MDSCs can be measured, e.g., as the absolute number in peripheral blood, e.g., measured in cells per μl, or as the percent of a total cell population in peripheral blood, e.g., the percentage of mononuclear cells or the percentage polymorphonuclear cells, that are MDSCs. The number of MDSCs can also be measured (either as total cells or as the percentage of a population of cells) in the patient's tumor microenvironment. The MDSCs can be M-MDSCs, e.g., MDSCs with a CD14⁺, HLA-DR^{-/low}, CD11b⁺, CD33⁺, Ln⁻ phenotype, wherein Ln is a cocktail of markers that define non-MDSCs, e.g., one or more of CD3, CD19, and/or CD56.

In another embodiment, the disclosure is directed to the use of a SEMA4D antagonist, e.g., an anti-SEMA4D antibody or antigen-binding fragment, variant, or derivative thereof, either as single agents or in combination with at least one other immune modulating therapy, to treat cancer patients with levels of MDSCs in circulation prior to treatment that fall within or below the range of normal individuals. As used herein, the term “normal” refers to the levels of MDSCs, or any specific MDSC population, that is found in healthy, non-cancer patients. As used herein, the term “within” refers to a ten (10) percent difference in the MDSC levels. Of course, one skilled in the art will appreciate that the levels of MDSCs can vary depending on a variety of factors, e.g., type of cancer, stage of cancer, etc., and, therefore, levels that are above the ones provided above can also constitute reduced levels for a certain type or stage of cancer. The number of MDSCs can be measured, e.g., as the absolute number in peripheral blood, e.g., measured in cells per μl, or as the percent of a total cell population in peripheral blood, e.g., the percentage of mononuclear cells or the percentage polymorphonuclear cells, that are MDSCs. The number of MDSCs can also be measured (either as total cells or as the percentage of a population of cells) in the patient's tumor microenvironment. The MDSCs can be M-MDSCs, e.g., MDSCs with a CD14⁺, HLA-DR^{-/low}, CD11b⁺, CD33⁺, Ln⁻ phenotype, wherein Ln is a cocktail of markers that define non-MDSCs, e.g., one or more of CD3, CD19, and/or CD56. In some embodiments, the absolute or relative MDSC cell counts can be measured using an immunophenotypic assay such as a standard flow cytometric-based immunophenotypic assay.

The methods described herein are applicable to any SEMA4D antagonists, including, e.g., anti-Plexin-B1 antibodies or antigen-binding fragments thereof, where the anti-Plexin-B1 antibody can be used to inhibit the interaction of SEMA4D with Plexin-B1 by blocking binding of SEMA4D to Plexin-B1 and/or by preventing activation of Plexin-B1 by SEMA4D. The methods described herein are also applicable to the use of small molecule SEMA4D antagonists or other biologic products to inhibit the activity of SEMA4D or Plexin-B1. In some embodiments, a small molecule drug or a biologic product other than an anti-SEMA4D binding molecule can be used to inhibit the interaction of SEMA4D with Plexin-B1 by blocking binding of SEMA4D to Plexin-B1 and/or by preventing activation of Plexin-B1 by SEMA4D.

In one embodiment, treatment includes the application or administration of an anti-SEMA4D antibody or antigen binding fragment thereof as described herein as a single agent or in combination with at least one other immune modulating therapy to a patient, or application or administration of the anti-SEMA4D antibody as a single agent or in combination with at least one other immune modulating therapy to an isolated tissue or cell line from a patient, where the patient has, or has the risk of developing metastases of cancer cells. In certain aspects the patient, prior to treatment, has a reduced level of MDSCs, e.g., below a predetermined threshold level. In another embodiment, treatment is also intended to include the application or administration of a pharmaceutical composition comprising the anti-SEMA4D antibody or antigen binding fragment thereof to a patient, in combination with at least one other immune modulating therapy or application or administration of a pharmaceutical composition comprising the anti-SEMA4D antibody and at least one other immune modulating therapy to an isolated tissue or cell line from a patient, where the patient has, or has the risk of developing metastases of cancer cells.

The anti-SEMA4D antibodies or binding fragments thereof as described herein, as single agents or in combination with at least one other immune modulating therapy are useful for the treatment of various malignant and non-malignant tumors. In certain aspects the patient, prior to treatment, has a reduced level of MDSCs, e.g., below a predetermined threshold level. By “anti-tumor activity” is intended a reduction in the rate of SEMA4D production or accumulation associated directly with the tumor or indirectly with stromal cells of the tumor environment, and hence a decline in growth rate of an existing tumor or of a tumor that arises during therapy, and/or destruction of existing neoplastic (tumor) cells or newly formed neoplastic cells, and hence a decrease in the overall size of a tumor and/or the number of metastatic sites during therapy. For example, therapy with at least one anti-SEMA4D antibody as a single agent or in combination with at least one other immune modulating therapy causes a physiological response, for example, a reduction in metastases, that is beneficial with respect to treatment of disease states associated with SEMA4D-expressing cells in a human.

In one embodiment, the disclosure relates to the use of anti-SEMA4D antibodies or antigen-binding fragments, variants, or derivatives thereof, as a single agent or in combination with at least one other immune modulating therapy as a medicament, in the treatment or prophylaxis of cancer or for use in a precancerous condition or lesion to inhibit, reduce, prevent, delay, or minimalize the growth or metastases of tumor cells. In certain aspects the patient, prior to treatment, has a reduced level of MDSCs, e.g., below a predetermined threshold level.

In accordance with the methods of the present disclosure, at least one anti-SEMA4D binding molecule, e.g., an antibody or antigen binding fragment, variant, or derivative thereof, as a single agent or in combination with at least one other immune modulating therapy can be used to promote a positive therapeutic response with respect to a malignant human cell. By “positive therapeutic response” with respect to cancer treatment is intended an improvement in the disease in association with the anti-tumor activity of these binding molecules, e.g., antibodies or fragments thereof, and/or an improvement in the symptoms associated with the disease. In particular, the methods provided herein are directed to inhibiting, preventing, reducing, alleviating, delaying, or lessening growth of a tumor and/or the development of metastases of primary tumors in a patient. That is the prevention of distal tumor outgrowths, can be observed. Thus, for example, an improvement in the disease can be characterized as a complete response. By “complete response” is intended an absence of clinically detectable metastases with normalization of any previously abnormal radiographic studies, e.g. at the site of the primary tumor or the presence of tumor metastases in bone marrow. Alternatively, an improvement in the disease can be categorized as being a partial response. By “partial response” is intended at least about a 50% decrease in all measurable metastases (i.e., the number of tumor cells present in the subject at a remote site from the primary tumor). Alternatively, an improvement in the disease can be categorized as being relapse free survival or “progression free survival.” By “relapse free survival” is intended the time to recurrence of a tumor at any site. “Progression free survival” is the time before further growth of tumor at a site being monitored can be detected.

Inhibition, delay, or reduction of metastases can be assessed using screening techniques such as imaging, for example, fluorescent antibody imaging, bone scan imaging, and tumor biopsy sampling including bone marrow aspiration (BMA), or immunohistochemistry. In addition to these positive therapeutic responses, the subject undergoing therapy with the anti-SEMA4D binding molecule, e.g., an antibody or antigen-binding fragment, variant, or derivative thereof, can experience the beneficial effect of an improvement in the symptoms associated with the disease.

Clinical response can be assessed using screening techniques such as magnetic resonance imaging (MRI) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, RIA, chromatography, and the like.

To apply the methods and systems of the disclosure in certain embodiments, samples from a patient can be obtained before or after the administration of a therapy comprising an effective amount of a SEMA4D antagonist, e.g., an isolated antibody or antigen-binding fragment thereof that specifically binds to SEMA4D either alone or in combination with an effective amount of at least one other immune modulating therapy; to a subject having a solid tumor or a hematologic cancer. Samples can be screened for certain biomarkers, e.g., MDSC levels according to the methods provided elsewhere herein. In some cases, successive samples can be obtained from the patient after therapy has commenced or after therapy has ceased, and such samples can likewise be screened for certain biomarkers, e.g., MDSC levels. Samples can, for example, be requested by a healthcare provider (e.g., a doctor) or healthcare benefits provider, obtained and/or processed by the same or a different healthcare provider (e.g., a nurse, a hospital) or a clinical laboratory, and after processing, the results can be forwarded to yet another healthcare provider, healthcare benefits provider or the patient. Similarly, the measuring/determination of one or more scores, comparisons between scores, evaluation of the scores and treatment decisions can be performed by one or more healthcare providers, healthcare benefits providers, and/or clinical laboratories.

As used herein, the term “healthcare provider” refers to individuals or institutions that directly interact and administer to living subjects, e.g., human patients. Non-limiting examples of healthcare providers include doctors, nurses, technicians, therapist, pharmacists, counselors, alternative medicine practitioners, medical facilities, doctor's offices, hospitals, emergency rooms, clinics, urgent care centers, alternative medicine clinics/facilities, and any other entity providing general and/or specialized treatment, assessment, maintenance, therapy, medication, and/or advice relating to all, or any portion of, a patient's state of health, including but not limited to general medical, specialized medical, surgical, and/or any other type of treatment, assessment, maintenance, therapy, medication and/or advice.

In some aspects, a healthcare provider can administer or instruct another healthcare provider to administer a therapy comprising an effective amount of a SEMA4D antagonist, e.g., an isolated antibody or antigen-binding fragment thereof that specifically binds to SEMA4D either alone or in combination with an effective amount of at least one other immune modulating therapy, where the subject has, or is suspected to have cancer. A healthcare provider can implement or instruct another healthcare provider or patient to perform the following actions: obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, administer a therapy (e.g., an effective amount of a SEMA4D antagonist, e.g., an isolated antibody or antigen-binding fragment thereof that specifically binds to SEMA4D either alone or in combination with an effective amount of at least one other immune modulating therapy to a subject, where the subject has, or is suspected to have cancer, commence the administration of a therapy, cease the administration of a therapy, continue the administration of a therapy, temporarily interrupt the administration of a therapy, increase the amount of an administered therapeutic agent, decrease the amount of an administered therapeutic agent, continue the administration of an amount of a therapeutic agent, increase the frequency of administration of a therapeutic agent, decrease the frequency of administration of a therapeutic agent, maintain the same dosing frequency on a therapeutic agent, replace a therapy or therapeutic agent by at least another therapy or therapeutic agent, combine a therapy or therapeutic agent with at least another therapy or additional therapeutic agent. In some aspects, a healthcare benefits provider can authorize or deny, for example, collection of a sample, processing of a sample, submission of a sample, receipt of a sample, transfer of a sample, analysis or measurement a sample, quantification a sample, provision of results obtained after analyzing/measuring/quantifying a sample, transfer of results obtained after analyzing/measuring/quantifying a sample, comparison/scoring of results obtained after analyzing/measuring/quantifying one or more samples, transfer of the comparison/score from one or more samples, administration of a therapy or therapeutic agent, commencement of the administration of a therapy or therapeutic agent, cessation of the administration of a therapy or therapeutic agent, continuation of the administration of a therapy or therapeutic agent, temporary interruption of the administration of a therapy or therapeutic agent, increase of the amount of administered therapeutic agent, decrease of the amount of administered therapeutic agent, continuation of the administration of an amount of a therapeutic agent, increase in the frequency of administration of a therapeutic agent, decrease in the frequency of administration of a therapeutic agent, maintain the same dosing frequency on a therapeutic agent, replace a therapy or therapeutic agent by at least another therapy or therapeutic agent, or combine a therapy or therapeutic agent with at least another therapy or additional therapeutic agent.

In addition, a healthcare benefits provides can, e.g., authorize or deny the prescription of a therapy, authorize or deny coverage for therapy, authorize or deny reimbursement for the cost of therapy, determine or deny eligibility for therapy, etc.

In some aspects, a clinical laboratory can, for example, collect or obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, or other related activities.

### Methods of Diagnosis and Treatment

In certain embodiments, this disclosure provides methods of treating a subject, e.g., a cancer patient, where the subject has MDSC levels below a predetermined threshold level, comprising administering a SEMA4D antagonists, e.g., an anti-SEMA4D antibody or antigen-binding fragment, variant, or derivative thereof either alone, or in combination with at least one other immune modulating agent as provided elsewhere herein, if the subject's MDSC level is below a predetermined threshold level or is equivalent or lower than the MDSC level in one or more control samples that can include, but are not limited to, samples from other cancer patients or from healthy, non-cancer patients. MDSC levels, either absolute levels or the percentage of another cell population, can be measured by a healthcare provider or by a clinical laboratory, where a sample, e.g., a blood sample or tumor biopsy, is obtained from the patient either by the healthcare provider or by the clinical laboratory. In one aspect, the patient's MDSC level can be measured in an immunophenotyping assay, such as a cytometric-based immunophenotypic assay.

This disclosure also provides methods, assays, and kits to facilitate a determination by a healthcare provider, a healthcare benefits provider, or a clinical laboratory to as to whether a subject, e.g., a cancer patient, will benefit from treatment with an effective amount of a SEMA4D antagonist, e.g., an isolated antibody or antigen-binding fragment thereof that specifically binds to SEMA4D either alone or in combination with an effective amount of at least one other immune modulating therapy, where the subject has, or is suspected to have cancer. The methods, assays, and kits provided herein will also facilitate a determination by a healthcare provider, a healthcare benefits provider, or a clinical laboratory to as to whether a subject, e.g., a cancer patient, will benefit from treatment with an effective amount of a SEMA4D antagonist, e.g., an isolated antibody or antigen-binding fragment thereof that specifically binds to SEMA4D either alone or in combination with an effective amount of at least one other immune modulating therapy.

The present disclosure provides a method of treating a subject, e.g., a cancer patient, comprising administering an effective amount of a SEMA4D antagonist, e.g., an isolated antibody or antigen-binding fragment thereof that specifically binds to SEMA4D either alone or in combination with an effective amount of at least one other immune modulating therapy if the level of MDSCs in a sample taken from the patient prior to treatment is below a predetermined threshold level, or is below or equivalent to the MDSC levels in one or more control samples. In certain aspects, the sample is obtained from the patient and is submitted for measurement of the level MDSCs in the sample, for example, to a clinical laboratory.

Also provided is a method of treating a subject, e.g., a cancer patient, s comprising (a) submitting a sample taken from the subject for measurement of MDSC levels in the sample; and, (b) administering an effective amount of a SEMA4D antagonist, e.g., an isolated antibody or antigen-binding fragment thereof that specifically binds to SEMA4D either alone or in combination with an effective amount of at least one other immune modulating therapy to the subject if the subject's MDSC level is below a predetermined threshold level, or is below or equivalent to the MDSC levels in one or more control samples.

The disclosure also provides a method of treating a subject, e.g., a cancer patient, comprising (a) measuring the level of MDSCs in a sample obtained from a subject, e.g., a cancer patient, wherein the subject's level of MDSCs in the sample is measured, e.g., in a cytometric-based immunophenotypic assay; (b) determining whether the level of MDSCs in the sample is below a predetermined threshold level, or is below or equivalent to the level of MDSCs in one or more control samples; and, (c) advising, instructing, or authorizing a healthcare provider to administer an effective amount of a SEMA4D antagonist, e.g., an isolated antibody or antigen-binding fragment thereof that specifically binds to SEMA4D either alone or in combination with an effective amount of at least one other immune modulating therapy to the subject if the subject's level of MDSCs is below a predetermined threshold level, or is below or equivalent the level of MDSCs in one or more control samples.

In certain aspects, the subject's level of MDSCs can be measured in a cytometric-based immunophenotypic assay. In certain aspects, the assay can be performed on a sample obtained from the subject, by the healthcare professional treating the patient, e.g., using an assay as described herein, formulated as a “point of care” diagnostic kit. In certain aspects, a sample can be obtained from the subject and can be submitted, e.g., to a clinical laboratory, for measurement of the level of MDSCs in the sample according to the healthcare professional's instructions, including but not limited to, using a cytometric-based immunophenotypic assay as described herein. In certain aspects, the clinical laboratory performing the assay can advise the healthcare provider or a healthcare benefits provider as to whether the subject can benefit from treatment with an effective amount of an isolated binding molecule that specifically binds to semaphorin-4D (SEMA4D) and an effective amount of at least one other immune modulating therapy, if the subject's level of MDSCs is below a predetermined threshold level, or is below or equivalent the level of MDSCs in one or more control samples.

In certain aspects, results of an immunoassay as provided herein can be submitted to a healthcare benefits provider for determination of whether the patient's insurance will cover treatment with an effective amount of a SEMA4D antagonist, e.g., an isolated antibody or antigen-binding fragment thereof that specifically binds to SEMA4D either alone or in combination with an effective amount of at least one other immune modulating therapy.

### Pharmaceutical Compositions and Administration Methods

Methods of preparing and administering SEMA4D antagonists, e.g., anti-SEMA4D antibodies, or antigen-binding fragments, variants, or derivatives thereof as a single agent or in combination with at least one other immune modulating therapy to a subject in need thereof are well known to or are readily determined by those skilled in the art. The route of administration of the SEMA4D antagonist, e.g., the isolated antibody or antigen-binding fragment thereof that specifically binds to SEMA4D either alone or in combination with an effective amount of at least one other immune modulating therapy, can be, for example, oral, parenteral, by inhalation or topical at the same or different times for each therapeutic agent. The term parenteral as used herein includes, e.g., intravenous, intraarterial, intraperitoneal, intramuscular, subcutaneous, rectal, or vaginal administration. While all these forms of administration are clearly contemplated as being within the scope of the disclosure, an example of a form for administration would be a solution for injection, in particular for intravenous or intraarterial injection or drip. A suitable pharmaceutical composition for injection can comprise a buffer (e.g. acetate, phosphate or citrate buffer), a surfactant (e.g. polysorbate), optionally a stabilizer agent (e.g. human albumin), etc. However, in other methods compatible with the teachings herein, SEMA4D antagonists, e.g., isolated antibodies or antigen-binding fragments thereof that specifically binds to SEMA4D either alone or in combination with an effective amount of at least one other immune modulating therapy can be delivered directly to the site of the adverse cellular population thereby increasing the exposure of the diseased tissue to the therapeutic agent.

As discussed herein, SEMA4D antagonists, e.g., isolated antibodies or antigen-binding fragments thereof that specifically binds to SEMA4D either alone or in combination with an effective amount of at least one other immune modulating therapy can be administered in a pharmaceutically effective amount for the in vivo treatment of diseases such as neoplastic disorders, including solid tumors. The disclosed agents can be formulated so as to facilitate administration and promote stability of the active agent. In certain embodiments, pharmaceutical compositions in accordance with the present disclosure comprise a pharmaceutically acceptable, non-toxic, sterile carrier such as physiological saline, non-toxic buffers, preservatives and the like. For the purposes of the instant application, a pharmaceutically effective amount of a SEMA4D antagonist, e.g., an isolated antibody or antigen-binding fragment thereof that specifically binds to SEMA4D either alone or in combination with an effective amount of at least one other immune modulating therapy shall be held to mean an amount sufficient to achieve effective binding to a target and to achieve a benefit, i.e., to inhibit, delay, or reduce metastases in a cancer patient.

The pharmaceutical compositions used in this disclosure comprise pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include, e.g., water, alcoholic/aqueous solutions, emulsions or suspensions, including saline and buffered media. Pharmaceutically acceptable carriers can include, but are not limited to, 0.01-0.1 M, or 0.05 M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as, for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition can be sterile and should be fluid to the extent that easy syringability exists. It should be stable under the conditions of manufacture and storage and can be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of a certain particle size in the case of dispersion and by the use of surfactants. Suitable formulations for use in the therapeutic methods disclosed herein are described in Remington's Pharmaceutical Sciences (Mack Publishing Co.) 21st ed. (2005).

Prevention of the action of microorganisms can be achieved by various antibacterial and antifungal agents, for example, parabens, chlorobutanol, phenol, ascorbic acid, thimerosal and the like. In certain embodiments, isotonic agents, for example, sugars, polyalcohols, such as mannitol, sorbitol, or sodium chloride can be included in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., an anti-SEMA4D antibody, or antigen-binding fragment, variant, or derivative thereof, by itself or in combination with at least one other immune modulating therapy) in a certain amount in an appropriate solvent with one or a combination of ingredients enumerated herein, followed by filtered sterilization. Generally, dispersions are prepared by incorporating the active compound into a sterile vehicle, which contains a basic dispersion medium and the other ingredients from those enumerated above. In the case of sterile powders for the preparation of sterile injectable solutions, methods of preparation can include vacuum drying or freeze-drying, which can yield a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations can be packaged and sold in the form of a kit. Such articles of manufacture can have labels or package inserts indicating that the associated compositions are useful for treating a subject suffering from or predisposed to a disease or disorder.

Parenteral formulations can be a single bolus dose, an infusion or a loading bolus dose followed with a maintenance dose. These compositions can be administered at specific fixed or variable intervals, e.g., once a day, or on an “as needed” basis.

Certain pharmaceutical compositions can be orally administered in an acceptable dosage form including, e.g., capsules, tablets, aqueous suspensions or solutions. Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

The amount of a SEMA4D antagonist, e.g., an isolated antibody or antigen-binding fragment thereof that specifically binds to SEMA4D either alone or in combination with an effective amount of at least one other immune modulating therapy to be combined with the carrier materials to produce a single dosage form will vary depending upon the host treated and the particular mode of administration. The composition can be administered as a single dose, multiple doses or over an established period of time in an infusion. Dosage regimens also can be adjusted to provide the optimum desired response (e.g., a therapeutic or prophylactic response).

In keeping with the scope of the present disclosure, SEMA4D antagonists, e.g., isolated antibodies or antigen-binding fragments thereof that specifically binds to SEMA4D either alone or in combination with an effective amount of at least one other immune modulating therapy can be administered to a human or other animal in accordance with the aforementioned methods of treatment in an amount sufficient to produce a therapeutic effect. The SEMA4D antagonists, e.g., isolated antibodies or antigen-binding fragments thereof that specifically binds to SEMA4D either alone or in combination with an effective amount of at least one other immune modulating therapy can be administered to such human or other animal in a conventional dosage form prepared by combining the antibody provided herein with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of anti-SEMA4D antibodies, or antigen-binding fragments, variants, or derivatives thereof as provided herein can be used.

By “therapeutically effective dose or amount” or “effective amount” is intended an amount of a SEMA4D antagonist, e.g., an isolated antibody or antigen-binding fragment thereof that specifically binds to SEMA4D either alone or in combination with an effective amount of at least one other immune modulating therapy, that when administered brings about a positive therapeutic response with respect to treatment of a patient with a disease to be treated, e.g., an inhibition, delay, or reduction of metastases in the patient.

Therapeutically effective doses of the compositions of the present disclosure, for the inhibition, delay, or reduction of tumor growth or metastases, vary depending upon many different factors, including means of administration, target site, physiological state of the patient, whether the patient is human or an animal, other medications administered, and whether treatment is prophylactic or therapeutic. In certain embodiments the patient is a human, but non-human mammals including transgenic mammals can also be treated. Treatment dosages can be titrated using routine methods known to those of skill in the art to optimize safety and efficacy.

The amount of SEMA4D antagonist, e.g., an isolated antibody or antigen-binding fragment thereof that specifically binds to SEMA4D either alone or in combination with an effective amount of at least one other immune modulating therapy is readily determined by one of ordinary skill in the art without undue experimentation given the disclosure of the present disclosure. Factors influencing the mode of administration and the respective amount therapeutic agent include but are not limited to, the severity of the disease, the history of the disease, the potential for metastases, and the age, height, weight, health, and physical condition of the individual undergoing therapy. Similarly, the amount of therapeutic agent to be administered will be dependent upon the mode of administration and whether the subject will undergo a single dose or multiple doses of this agent.

The disclosure also provides for the use of an effective amount of a SEMA4D antagonist, e.g., an isolated antibody or antigen-binding fragment thereof that specifically binds to SEMA4D either alone or in combination with an effective amount of at least one other immune modulating therapy in the manufacture of a medicament for treating a subject with a cancer. In certain aspects the medicament is used in a subject that has been pretreated with at least one other therapy. By “pretreated” or “pretreatment” is intended the subject has received one or more other therapies (e.g., been treated with at least one other cancer therapy) prior to receiving the medicament comprising the SEMA4D antagonist, e.g., the isolated antibody or antigen-binding fragment thereof that specifically binds to SEMA4D either alone or in combination with an effective amount of at least one other immune modulating therapy. “Pretreated” or “pretreatment” includes subjects that have been treated with at least one other therapy within 2 years, within 18 months, within 1 year, within 6 months, within 2 months, within 6 weeks, within 1 month, within 4 weeks, within 3 weeks, within 2 weeks, within 1 week, within 6 days, within 5 days, within 4 days, within 3 days, within 2 days, or even within 1 day prior to initiation of treatment with the medicament comprising the anti-SEMA4D antibody, for example, the monoclonal antibody pepinemab disclosed herein, or antigen-binding fragment, variant, or derivative thereof as a single agent or in combination with at least one other immune modulating therapy. It is not necessary that the subject was a responder to pretreatment with the prior therapy or therapies. Thus, the subject that receives the medicament comprising the SEMA4D antagonist, e.g., the isolated antibody or antigen-binding fragment thereof that specifically binds to SEMA4D either alone or in combination with an effective amount of at least one other immune modulating therapy could have responded, or could have failed to respond (e.g., the cancer was refractory), to pretreatment with the prior therapy, or to one or more of the prior therapies where pretreatment comprised multiple therapies. Examples of other cancer therapies for which a subject can have received pretreatment prior to receiving the medicament comprising the provided therapeutic agent include, but are not limited to, surgery; radiation therapy; chemotherapy, optionally in combination with autologous bone marrow transplant, where suitable chemotherapeutic agents include, but are not limited to, those listed herein above; other anti-cancer monoclonal antibody therapy; small molecule-based cancer therapy, including, but not limited to, the small molecules listed herein above; vaccine/immunotherapy-based cancer therapies; steroid therapy; other cancer therapy; or any combination thereof.

This disclosure employs, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Green and Sambrook, ed. (2012) Molecular Cloning A Laboratory Manual (4th ed.; Cold Spring Harbor Laboratory Press); Sambrook et al., ed. (1992) Molecular Cloning: A Laboratory Manual, (Cold Springs Harbor Laboratory, NY); D. N. Glover and B. D. Hames, eds., (1995) DNA Cloning 2d Edition (IRL Press), Volumes 1-4; Gait, ed. (1990) Oligonucleotide Synthesis (IRL Press); Mullis et al. U.S. Pat. No. 4,683,195; Hames and Higgins, eds. (1985) Nucleic Acid Hybridization (IRL Press); Hames and Higgins, eds. (1984) Transcription And Translation (IRL Press); Freshney (2016) Culture Of Animal Cells, 7th Edition (Wiley-Blackwell); Woodward, J., Immobilized Cells And Enzymes (IRL Press) (1985); Perbal (1988) A Practical Guide To Molecular Cloning; 2d Edition (Wiley-Interscience); Miller and Calos eds. (1987) Gene Transfer Vectors For Mammalian Cells, (Cold Spring Harbor Laboratory); S. C. Makrides (2003) Gene Transfer and Expression in Mammalian Cells (Elsevier Science); Methods in Enzymology, Vols. 151-155 (Academic Press, Inc., N.Y.); Mayer and Walker, eds. (1987) Immunochemical Methods in Cell and Molecular Biology (Academic Press, London); Weir and Blackwell, eds.; and in Ausubel et al. (1995) Current Protocols in Molecular Biology (John Wiley and Sons).

General principles of antibody engineering are set forth, e.g., in Strohl, W. R., and L. M. Strohl (2012), Therapeutic Antibody Engineering (Woodhead Publishing). General principles of protein engineering are set forth, e.g., in Park and Cochran, eds. (2009), Protein Engineering and Design (CDC Press). General principles of immunology are set forth, e.g., in: Abbas and Lichtman (2017) Cellular and Molecular Immunology 9th Edition (Elsevier). Additionally, standard methods in immunology known in the art can be followed, e.g., in Current Protocols in Immunology (Wiley Online Library); Wild, D. (2013), The Immunoassay Handbook 4th Edition (Elsevier Science); Greenfield, ed. (2013), Antibodies, a Laboratory Manual, 2d Edition (Cold Spring Harbor Press); and Ossipow and Fischer, eds., (2014), Monoclonal Antibodies: Methods and Protocols (Humana Press).

All of the references cited above, as well as all references cited herein, are incorporated herein by reference in their entireties.

The following examples are offered by way of illustration and not by way of limitation.

### EXAMPLES

### Example 1: MDSC Levels as a Biomarker for SEMA4D-Based Cancer Immunotherapy

Blockade of the PD-1/PD-L1 pathway is an effective immunotherapy for NSCLC, however rational combination therapies are needed to overcome resistance mechanisms. The CLASSICAL-Lung clinical trial is testing the combination of pepinemab with avelumab to couple immune activation via checkpoint inhibition with beneficial modifications of the tumor immune microenvironment via pepinemab.

A phase 1b/2, open label, single arm, first-in-human combination study is currently in progress to evaluate the safety, tolerability and efficacy of pepinemab in combination with avelumab in 62 subjects with advanced (IIIB/IV) NSCLC.

Pepinemab (VX15/2503) is an IgG4 humanized monoclonal antibody targeting semaphorin 4D (SEMA4D, CD100). The VH comprises the amino acid sequence SEQ ID NO: 1 and the VL comprises the amino acid sequence SEQ ID NO: 5. In vivo preclinical models demonstrated antibody blockade of SEMA4D promoted infiltration of CD8+ T cells and dendritic cells, and reduced function and recruitment of immunosuppressive myeloid and regulatory T cells (Treg) within the tumor. Importantly, preclinical combinations of anti-SEMA4D with various immunotherapies enhanced T cell activity and tumor regression. See, e.g., U.S. Pat. No. 9,243,068, which is incorporated herein by reference in its entirety.

Avelumab Is a fully human anti-PD-L1 IgG1 antibody that has been approved for the treatment of both Merkel cell and urothelial carcinomas. Avelumab inhibits PD-L1-PD-1 interactions and also has the potential to induce ADCC. The heavy and light chain of avelumab are presented as SEQ ID NO: 11 and SEQ ID NO: 12.

### Study Design

The trial is split into dose escalation (n=12) and dose expansion (n=50) phases. The dose escalation portion includes patients who are immunotherapy naïve and have either progressed or declined standard first or second-line systemic anticancer therapy. Patients in the dose escalation cohorts received ascending doses of pepinemab (5, 10, 20 mg/kg, Q2W) in combination with avelumab (10 mg/kg, Q2W).

The expansion phase includes a similar patient cohort as well as a second cohort of patients whose tumors progressed during or following immunotherapy.

### Demographic Characteristics

All subjects presented at baseline with stage IV carcinoma. There was an even distribution of adenocarcinoma and squamous cell carcinoma subjects. Sixty-seven percent of subjects received prior systemic treatment.

Correlations of Baseline Levels of Immune Cells with Time on Study

Initial analysis of peripheral blood immune cell subsets at baseline versus weeks on study suggests that higher levels of T cells and lower levels of MDSCs correlate with length of time on study.

Prior to treatment, subjects were evaluated for initial levels of CD8+ T cells and CD14⁺, HLA-DR^{low}, CD11b⁺, CD33⁺, Ln⁻ phenotype MDSC cells. “Ln” is a group of makers that were ruled out the cell population and included CD3, CD19, CD56. “Days on Study” at this preliminary point of the study is based on death, voluntary withdrawal or disease progression. Spearman rank-order correlation between cell subsets at baseline and weeks on study. The cutoff for correlation graphs was Jan. 28, 2019.

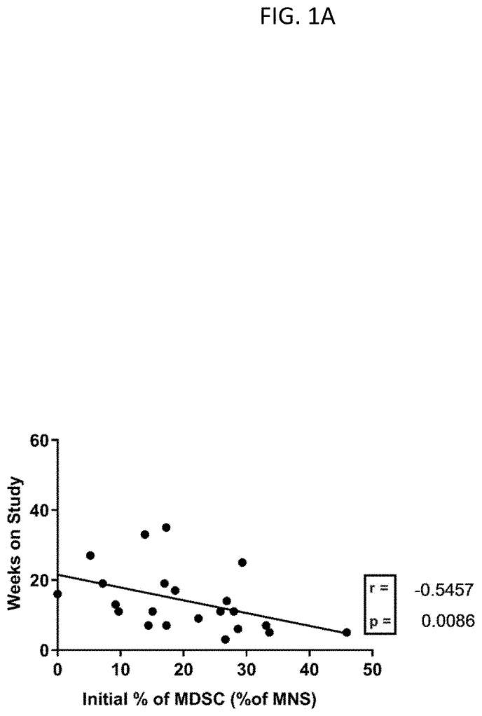

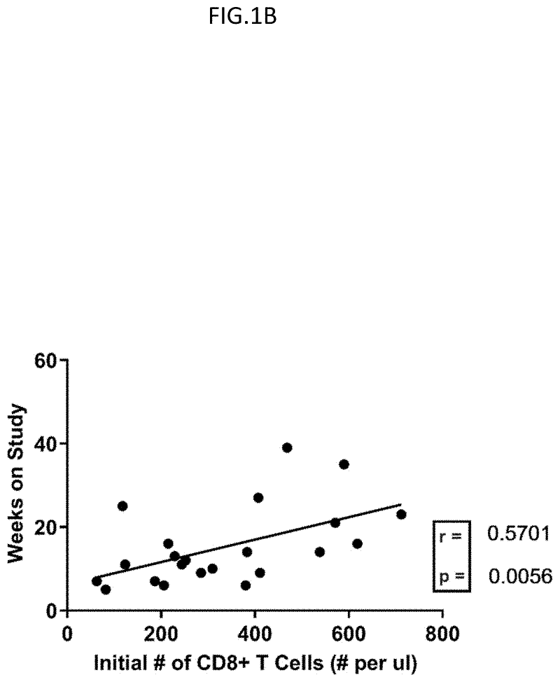

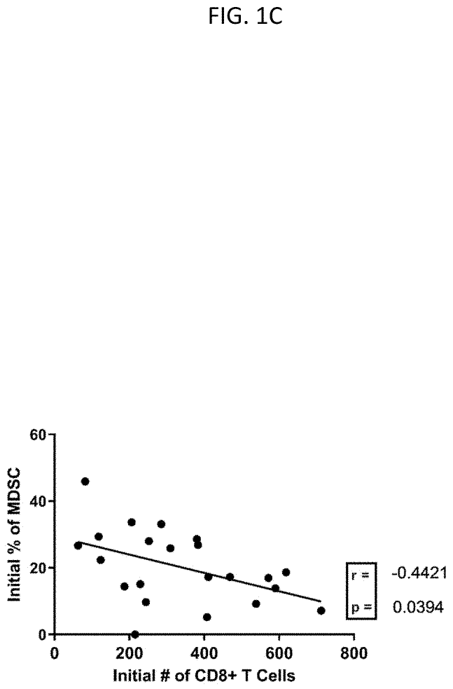

Either absolute or 0% cell subsets in peripheral blood were measured at baseline by flow cytometry at a central lab. The number of weekly pepinemab doses administered until disease progression is plotted versus the absolute (cells/μl) (FIG. 1A, for CD8+ T cells) or % of MDSC (of mononuclear cells) (FIG. 1B) of peripheral blood subsets at baseline (average of a screening visit and baseline visit). FIG. 1C plots the percent of initial CD8+ T cells versus initial VMDSCs in peripheral blood. The respective Spearman rank-order correlation coefficients (r) and p values for each analysis are provided. “Weeks on study” is defined as time from first dose to end of treatment or cut-off date for analysis, Jan. 25, 2019.

TABLE 2
Sequences

| SEQ | | |
|---|---|---|
| ID NO | Description | Sequence |
| 1 | VX15/2503 | QVQLVQSGAEVKKPGSSVKVSCKASGYSFSDYYMHWVRQA |
| | VH | PGQGLEWMGQINPTTGGASYNQKFKGKATITVDKSTSTAYM |
| | | ELSSLRSEDTAVYYCARYYYGRHFDVWGQGTTVTVSS |
| 2 | VX15/2503 | GYSFSDYYMH |
| | HCDR1 | |
| 3 | VX15/2503 | QINPTTGGASYNQKFKG |
| | HCDR2 | |
| 4 | VX15/2503 | YYYGRHFDV |
| | HCDR3 | |
| 5 | VX15/2503 | DIVMTQSPDSLAVSLGERATINCKASQSVDYDGDSYMNWYQ |
| | VL | QKPGQPPKLLIYAASNLESGVPDRFSGSGSGTDFTLTISSLQAE |
| | | DVAVYYCQQSNEDPYTFGQGTKLEIK |
| 6 | VX15/2503 | KASQSVDYDGDSYMN |
| | LCDR1 | |
| 7 | VX15/2503 | AASNLES |
| | LCDR2 | |
| 8 | VX15/2503 | QQSNEDPYT |
| | LCDR3 | |
| 9 | Mab 67 VH | QVQLQQSGPELVKPGASVKISCKASGYSFSDYYMHWVKQSP |
| | | ENSLEWIGQINPTTGGASYNQKFKGKATLTVDKSSSTAYMQL |
| | | KSLTSEESAVYYCTRYYYGRHFDVWGQGTTVTVSS |
| 10 | Mab 67 VL | DIVMTQSPASLAVSLGQRATISCKASQSVDYDGDSYMNWYQ |
| | | QKPGQPPKLLIYAASNLESGIPARFSGSGSGTDFTLNIHPVEEE |
| | | DAATYYCQQSNEDPYTFGGGTKLEIK |
| 11 | Avelumab | EVQLLESGGGLVQPGGSLRLSCAASGFTFSSYIMMWVRQAPGKGLEWVSSIYPSGGIT |
| | heavy | FYADTVKGRFTISRDNSKNTLYLQMNSLRAEDTAVYYCARIKLGTVTTVDYWGQGTLV |
| | chain | TVSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKKVEPKSCDKTHTCPPC |
| | | PAPELLGGPSVFLFPPKPKDTLMISRTPEVTCVVVDVSHEDPEVKFNWYVDGVEVHNA |
| | | KTKPREEQYNSTYRWSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLDSDGS |
| | | FFLYSKLTVDKSRWQQGNVFSCSVMHEALHNHYTQKSLSLSPGK |
| 12 | Avelumab | QSALTQPASVSGSPGQSITISCTGTSSDVGGYNYVSWYQQHPGKAPKLMIYDVSNRPS |
| | light | GVSNRFSGSKSGNTASLTISGLQAEDEADYYCSSYTSSSTRVFGTGTKVTVLGQPKAN |
| | chain | PTVTLFPPSSEELQANKATLVCLISDFYPGAVTVAWKADGSPVKAGVETTKPSKQSNN |
| | | KYAASSYLSLTPEQWKSHRSYSCQVTHEGSTVEKTVAPTECS |

What is claimed is:

1. A method for treating, inhibiting, delaying, or reducing malignant cell growth in a subject with cancer and in need of treatment, comprising:
(a) determining the subject's level of circulating myeloid-derived suppressor cells (MDSCs) by;
(b) obtaining or having obtained a biological sample from the subject;
(c) performing or having performed an assay on the biological sample to determine the level of MDSCs in the biological sample; and
(d) administering to the subject an effective amount of a cancer immunotherapy regimen comprising an isolated antibody or antigen-binding fragment thereof that specifically binds to semaphorin-4D (SEMA4D) if the level of MDSCs is below a predetermined threshold level, thereby treating the subject.

2. The method of claim [076], wherein the anti-SEMA4D antibody or fragment thereof inhibits SEMA4D interaction with its receptor.

3. The method of claim [079], wherein the receptor is Plexin-B1, Plexin-B2, CD72, or any combination thereof.

4. The method of claim 3, wherein the antibody or fragment thereof inhibits SEMA4D-mediated signal transduction.

5. The method of claim [076], wherein the antibody or fragment thereof comprises a variable heavy chain (VH) comprising VH CDRs 1-3 comprising SEQ ID NOS: 2, 3, and 4, respectively, and a variable light chain (VL) comprising VL CDRs 1-3 comprising SEQ ID NOS: 6, 7, and 8, respectively.

6. The method of claim 5, wherein the VH and VL comprise, respectively, SEQ ID NO: 1 and SEQ ID NO: 5, or SEQ ID NO: 9 and SEQ ID NO: 10.

7. The method of claim [076], wherein the cancer immunotherapy regimen further comprises an additional cancer immunotherapy agent.

8. The method of claim [080], wherein the additional cancer immunotherapy agent comprises an immune checkpoint blockade.

9. The method of claim [080], wherein the agent that inhibits an immune checkpoint blockade comprises an antibody or antigen-binding fragment thereof that specifically binds to CTLA4, PD-1, PD-L1, LAG3, TIM3, B7-H3, or any combination thereof.

10. The method of claim [080], wherein the antibody or antigen-binding fragment thereof comprises the anti-PD-L1 antibody Avelumab.

11. The method of claim [076], wherein the MDSCs are mononuclear MDSCs (M-MDSCs).

12. The method of claim [077], wherein the M-MDSCs comprise a CD14, HLA-DR^{-/low}, CD11b⁺, CD33⁺, Ln⁻ phenotype, wherein Ln is a cocktail of markers that define non-MDSCs.

13. The method of claim [078], wherein the Ln markers comprise one or more of CD3, CD19, or CD56.

14. The method of claim [076], wherein the predetermined threshold level of MDSCs comprises less than 50%, less than 40%, less than 30%, less than 20%, or less than 10% of the subject's total peripheral blood mononuclear cells prior to treatment.

15. The method of claim 1, wherein the immunotyping assay is a flow cytometric-based immunophenotypic assay.

16. The method of claim 1, wherein the biological sample is a blood sample or a tumor biopsy sample.

17. The method of claim [076], wherein the cancer comprises a solid tumor, a hematological malignancy, any metastasis thereof, or any combination thereof.

18. The method of claim [081]7, wherein the cancer is a solid tumor or metastasis thereof.

19. The method of claim 188, wherein the solid tumor is a sarcoma, a carcinoma, a melanoma, any metastases thereof, or any combination thereof.

20. The method of claim [081]8, wherein the solid tumor is squamous cell carcinoma, adenocarcinoma, basal cell carcinoma, renal cell carcinoma, ductal carcinoma of the breast, soft tissue sarcoma, osteosarcoma, melanoma, small-cell lung cancer, non-small cell lung cancer, adenocarcinoma of the lung, cancer of the peritoneum, hepatocellular carcinoma, gastrointestinal cancer, gastric cancer, pancreatic cancer, neuroendocrine cancer, glioblastoma, cervical cancer, ovarian cancer, liver cancer, bladder cancer, brain cancer, hepatoma, breast cancer, colon cancer, colorectal cancer, endometrial or uterine carcinoma, esophageal cancer, salivary gland carcinoma, kidney cancer, liver cancer, prostate cancer, vulval cancer, thyroid cancer, head and neck cancer, any metastases thereof, or any combination thereof.

21. The method of claim 20, wherein the solid tumor is non-small cell lung cancer.

22. The method of claim [081]7, wherein the cancer is a hematologic malignancy or metastasis thereof.

23. The method of claim 222, wherein the hematologic malignancy is leukemia, lymphoma, myeloma, acute myeloid leukemia, chronic myeloid leukemia, acute lymphocytic leukemia, chronic lymphocytic leukemia, hairy cell leukemia, Hodgkin lymphoma, non-Hodgkin lymphoma, multiple myeloma, any metastases thereof, or any combination thereof.

24. The method of claim [076], further comprising administration of an additional cancer therapy.

25. The method of claim [082]4, wherein the additional therapy comprises surgery, chemotherapy, radiation therapy, a cancer vaccine, administration of an immunostimulatory agent, adoptive T cell therapy, administration of a regulatory T cell (Treg) modulator, or any combination thereof.

26. A method for selecting a subject with cancer for treating, inhibiting, delaying, or reducing malignant cell growth in the subject with a cancer immunotherapy regimen comprising an isolated antibody or antigen-binding fragment thereof that specifically binds to semaphorin-4D (SEMA4D), the method comprising:
(a) determining the subject's level of circulating myeloid-derived suppressor cells (MDSCs) in a sample obtained from the subject; and
(b) selecting the subject for treatment if the level of MDSCs in the sample is below a predetermined threshold level.

27. The method of claim 26, wherein the anti-SEMA4D antibody or fragment thereof inhibits SEMA4D interaction with its receptor.

28. The method of claim 26, wherein the receptor is Plexin-B1, Plexin-B2, CD72, or any combination thereof.

29. The method of claim 27, wherein the antibody or fragment thereof inhibits SEMA4D-mediated signal transduction.

30. The method of claim 26, wherein the antibody or fragment thereof comprises a variable heavy chain (VH) comprising VH CDRs 1-3 comprising SEQ ID NOS: 2, 3, and 4, respectively, and a variable light chain (VL) comprising VL CDRs 1-3 comprising SEQ ID NOS: 6, 7, and 8, respectively.

31. The method of claim 26, wherein the predetermined threshold level of MDSCs comprises less than 50%, less than 40%, less than 30%, less than 20%, or less than 10% of the subject's total peripheral blood mononuclear cells prior to treatment.
